(19) United States
(12) Patent Application Publication
(10) Pub. No.: US 2007/0059318 A1
(43) Pub. Date: Mar. 15, 2007

(54) Lipid nano particulates containing antigens as cancer vaccines

(76) Inventors: Sathy V. Balu-Iyer, Amherst, NY (US); Richard B. Bankert, Eden, NY (US)

Correspondence Address: HODGSON RUSS LLP, ONE M & T PLAZA, SUITE 2000, BUFFALO, NY, 14203-2391, US

(21) Appl. No.: 11/504,386

(22) Filed: Aug. 15, 2006

Related U.S. Application Data
(60) Provisional application No. 60/708,408, filed on Aug. 15, 2005.

(51) Int. Cl. A61K 39/02; A61K 39/00; A61K 9/127

(52) U.S. Cl. 424/184.1; 424/450; 977/907; 424/234.1

(57) ABSTRACT

The present invention provides compositions and method for increasing the immunogenicity of antigens such as tumor antigens. The compositions comprise liposomes such that they are suitable for targeting denderitic cells. The compositions preferably comprise at least 50% liposomes which are less than 120 nm. The liposomes comprise a cationic lipid and phosphatidyl choline. The antigen is intercalated within or in the bilayer or covalently linked to the liposomal molecules.

This application claims priority to U.S. provisional application No. 60/708,408 filed on Aug. 15, 2005, the disclosure of which is incorporated herein by reference.

### BACKGROUND

The standard options for cancer therapy such as surgery, radiotherapy, and chemotherapy have debilitating and distressing side effects, destroying healthy tissues along with cancer cells. Chemotherapy often presents problems such as toxicity, immunosuppression and intrinsic drug resistance. Very frequently, it is found that the patients face a relapse even after the course of the treatment is supposedly complete. Approaches that can specifically activate the immune system to control the cancer growth have been the focus of cancer immunology. Antigens that are specifically expressed in cancer cells serve as viable targets for the design of cancer vaccines.

The development of therapeutic cancer vaccines offers distinct advantages over conventional chemotherapy. For example, targeting the antitumor immune response to critical tumor specific antigens offers specificity and minimal toxicity; the immune response mediated anti-tumor response operates by a distinct mechanism, circumventing the drug resistance often a complication with conventional chemotherapy; and the immunologic memory offers an opportunity for durable therapeutic effect that is reactivated at the onset of disease relapse. Thus, cancer vaccines offer potential future for both therapy and prevention of the disease.

In theory, the mode of action of a cancer vaccine is simple: the vaccine prompts the immune system to produce anti-tumor antibodies and cytotoxic T lymphocytes (killer T cells), which target, destroy, and eradicate malignant cells (1). The cellular arm of immune system utilizes CD8 and CD4 cells for killing of target cells. Of particular note is the role of CD8 cells (killer cells), which, when activated, directly kill target cells (2). The activation of CD8 cells is brought about by specific antigen presenting cells, which can present the antigen to CD8 cells in the context of the MHC-I (major histocompatibility Class-I) complex. The antigens presented by the MHC-I are usually 8-10 amino acid peptides derived from a larger protein (3). Several research groups have been actively involved in using MHC 1 restricted antigenic peptides for vaccinations. Examples include an HLA-1 restricted MAGE-3 peptide in metastatic melanoma and an HLA-2 restricted gp 100 peptide synthetic analog, also in melanoma. The antigenic sequence also involves mucin 1, carcino embryonic antigen (CEA) and HER 2 vaccine (4, 5).

With the identification of several antigenic peptides, clinical trials have been initiated to induce T-cell immunity. The outcome of these trials has been disappointing as the efficacy of these vaccines was very low. Despite the fact that T-cell responses (6) and some antitumor responses were observed, the immune responses were short lived (7). However, these trials provided insight into the optimal properties required for an efficacious vaccine. These include selecting an appropriate antigen, stimulating potent and durable response (adjuvant and targeting relevant antigen presenting cells (APCs), and strategies to avoid autoimmunity and immune evasion (6-8). Another reason for the failure could be the degradation and elimination of peptides resulting in inefficient uptake and processing by potent antigen presenting cells (9). In order to improve the efficacy of antigens, peptides have been formulated in particulate systems such as microspheres, liposomes, alum precipitates in combination with cytokines such as IL-2 and granulocyte colony stimulating factors (10-12).

Liposomes are made of one or more concentric phospholipid bilayers enclosing an aqueous compartment. Due to their molecular properties, antigens can be attached to the external surface, encapsulated within the internal aqueous spaces or reconstituted within the lipid bilayers of the liposomes (11, 13). Further, liposomes are rapidly taken up by macrophages (antigen presenting cells) and this uptake by macrophages has led to the use of liposomal peptide for vaccine applications. Liposomes have been shown to potentiate a broad array of humoral and cellular immune responses (11). The imunoadjuvant activity of Liposomes has been well studied and shown that it can stimulate antibody responses against liposome associated protein antigens (14).

Mechanistically, it is achieved by presenting the protein and peptide antigens into MHC Class II Pathway of phagocytic APC and thereby enhance induction of antibodies and antigen specific T cell proliferative response (15). Therefore, such presentation leads to both IgM and IgG antibody synthesis with induction of immunological memory. Liposomes are also capable of stimulating cellular immunity, including the induction of CTL activity. This is based on their ability to deliver antigens into the MHC class I pathway (16). Such approaches involve the efficient uptake of liposomes by APCs. Mostly, the phagocytosed liposomes were localized in endosomes or lysosomes of macrophages but not in the cytoplasm and do not gain access to the endoplasmic reticulum or to the Golgi apparatus, major cellular organelles that contain the MHC Class 1. This results in ineffective presentation of antigen in MHC I pathway. Further, preferential uptake of liposomes by resident macrophages (17) leads to rapid elimination and limits the use of liposomes for T-cell mediated vaccine purposes as they are not available for potent antigen presenting cells such as Dendritic cells, a principal stimulator of T- and B-cell responses.

Recent advances in immuno biology of dendritic cells (DCs) have led to the idea that exploitation of DCs is a rational way to improve the efficacy of vaccines (18). DCs are the most potent APCs for the induction of T-cell responses and are central to the induction of adaptive responses (19). DCs are involved in the induction of CD8 and CD4 responses via class I and II MHC molecules. Further, DCs can trigger the expansion of naïve T-cells and play a pivotal role in the immune response. Therefore, DCs constitute a prime target for vaccination strategy.

There are three stages in the matutration of DCs, immature, intermediate and matured DCs (20). Immature DC resides in peripheral tissues such as skin and possesses high internalization potential to effectively capture and process native protein antigen. Endocytosis mediates the antigen capturing in immature DC and involves receptor mediated endocytosis, macropinocytosis and phagocytosis. Then the immature DCs migrate to peripheral lymphoid organs through the formation of intermediate DCs that are characterized by high internalization and high MHC synthesis. A maturation process, characterized by IL-12 production and the up-regulation of MHC and co stimulatory molecules, is critical for initiation of primary T cell response.

A variety of receptors are expressed on the surface of DCs for receptor mediated endocytosis of the antigens, that includes Fc and family of C-type lectin receptors (20). The C-type lectin family is capable of clustering in clatherin coated pits and includes mannose receptor that can effectively process mannosylated antigens. These receptors are absent in immature DCs located in skin called Langerhan cells (LCs). LCs expresses langerin a C-type family of lectin that are linked to the formation of Bebeck granules that may play a role in the processing of antigens. Macropincytosis have been observed with DCs and it is not clear how this influences the down stream antigen processing. Phagocytosis of particulate matter has been observed in DCs. The uptake of bateria resulted in presentation of antigens on both class II and class I MHC that is associated with maturation of DCs (21).

In order to exploit the potent antigen presenting properties of DCs, antigen loading of DCs in vitro was developed as vaccination strategies. The DCs were pulsed with antigenic peptides and activated in vitro and were injected into recipients for in vivo response. The delivery of antigens by liposomes has been observed and the presence of mannosylated lipid in liposomes containing PC:PG:Cholesterol and Neisseria meningitidis type B antigen PorA, increased the interaction of liposomes with DCs (22). The presence of CpG DNA, unmethylated RRCGY sequence also increases the DC uptake of liposomes (23). Further coating of poly ethylene Glycol (PEG) of liposomes containing ovalbumin initiated CD8 mediated T-cell responses via immune processing by DC (17). Huang and his colleagues have examined the use of cationic lipid and protamine containing lipidic structures as gene vector for potent vaccine carrier (24, 25). By this method, the lipidic structures have enhanced the delivery of genes that encodes antigenic peptides in DC's for potent response.

Despite the fact that the interaction of liposomal antigen with DCs promotes T-cell responses, the efficacy of vaccines is still a major problem. One of the major limiting factors is the rapid uptake of antigens by macrophages that leads to inefficient processing and presentation of the antigen (17). Thus, the preferential uptake of antigens by DCs is very critical for efficacy of vaccines but is inefficient. The use of mannosylated antigens may be beneficial to target DCs, however, macrophages also express mannose receptors further complicating the effective targeting of DCs (20). Therefore, there continues to be need to develop more efficient means for antigen presentation for vaccine applications.

### SUMMARY OF THE INVENTION

The present invention provides compositions comprising liposomes. The liposomes of the present invention comprise a cationic lipid and a phosphatidyl choline. Sufficient antigen is intercalated within or between the bilayers, or is covalently linked so as to be exposed to the exterior for targeting DCs. In one embodiment, preferably at least 50% of the liposomes are less than 120 nm. Lipid nano particles of less than 120 nm are not likely to be taken up by macrophages. Thus, use of a lipid nano particles less than 120 nm in diameter will increase immune response relative to use of only an antigen by increasing antigen availability to antigen presenting cells (APCs), i.e. dendritic cells (DCs). The compositions of the present invention can be used for increasing the immune response to any antigen, particularly tumor antigens.

### BRIEF DESCRIPTION OF THE DRAWINGS

FIG. 1. Schematic representation of proposed molecular characteristics of lipid nanoparticulates. (The molecular dimensions are not to the scale.)

FIG. 2: (A) Morphology by negative stain transmission electron micrograph, (B) Topology studied by acrylamide quenching of Trp fluorescence of 1H6Ig associated with lipid nanoparticulate, and (C) size distribution by Quasi Elastic light scattering of lipid nanoparticulate.

FIG. 3. The T-cell (Interferon gamma) responses in BALB/c mice bearing tumor cells following immunization with soluble and LINAP loaded 1H6Ig.

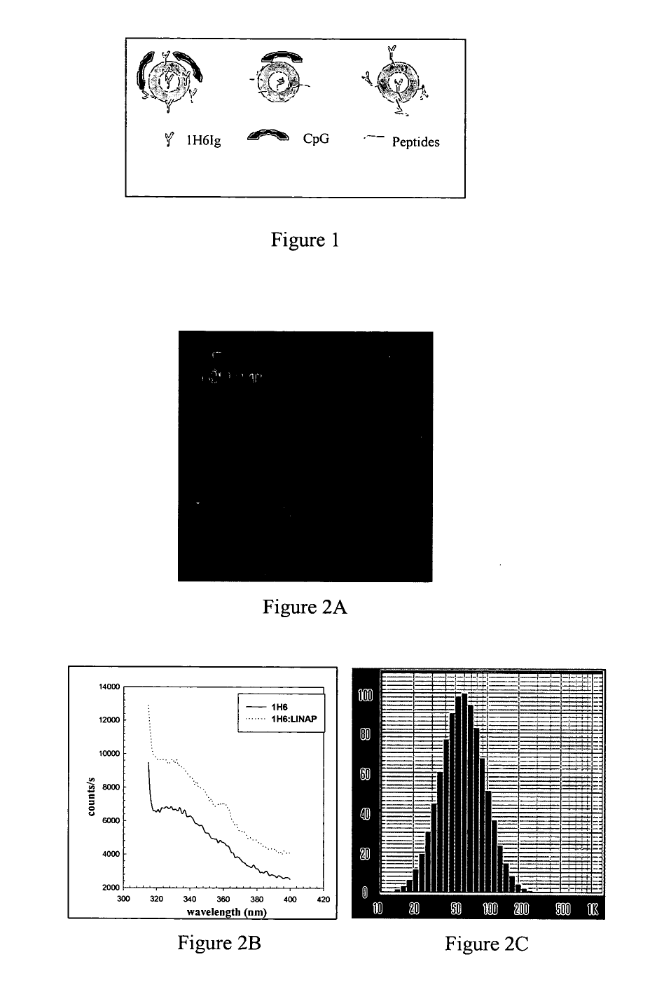

### DESCRIPTION OF THE INVENTION

The present invention comprises liposomes which are suitable for targeting dendritic cells (DCs). Thus, preferably, at least 50% of the liposomes are smaller than 120 nm (referred to herein as lipid nanoparticles). The composition of the present invention is suitable for targeting DCs. While not intending to be bound by any particular theory, it is believed that lipid nano particles can target DCs, but avoid uptake by macrophages in vivo. Because the uptake by macrophages is reduced, a decrease in the clearance of these lipid nano particles can be achieved. Further, this would effectively promote the availability of lipid nano particles in lymphoid tissue and other peripheral tissues where immature and intermediate DCs reside which possess high internalization characteristics suitable for antigen and particulate uptake. The uptake of lipid nano particles by DC cells is likely achieved by phagocytosis in addition to receptor mediated endocytosis and macropinocytosis.

The liposomes of the present invention comprise a cationic lipid, and a negatively charged phospholipid such as a phophatidyl choline (PC). Cationic lipids suitable for this invention will have acyl chains of 12-22 carbons. Examples of suitable cationic lipids include, but are not limited to, 1,2-Diacyl-3-Trimethylammonium-Propane (TAP); 1,2-Diacyl-3-Dimethylammonium-Propane (DAP); and 1,2-Diacyl-sn-Glycero-3-Ethylphosphocholine (EPC). The acyl chains of the cationic lipid may be saturated or unsaturated. In a preferred embodiment, the acyl chain is saturated. It is also preferable that the acyl chain is 16-22 carbons. Suitable examples of cationic lipids include 1,2-Dioleyl-3-Trimethylammonium-Propane (DOTAP); 1,2-Dioleyl-3-Dimethylammonium-Propane (DODAP). Other examples include 18:1 EPC, 18:0 EPC and 14:0-18:1 EPC.

The negatively charged phospholipids in the liposomes is preferably phosphatidyl choline. The acyl chains of the PC are 12-22 carbons in length and may be saturated or unsaturated.

Some non-limiting examples of 12-22 carbon atoms acyl chains for the cationic lipid and PC are shown in Tables 1A and 1B.

TABLE 1A

| Symbol | Common Name | Systematic name | Structure |
|---|---|---|---|
| 12:0 | Lauric acid | dodecanoic acid | CH₃(CH₂)₁₀COOH |
| 14:0 | Myristic acid | tetradecanoic acid | CH₃(CH₂)₁₂COOH |
| 16:0 | Palmitic acid | hexadecanoic acid | CH₃(CH₂)₁₄COOH |
| 18:0 | Stearic acid | octadecanoic acid | CH₃(CH₂)₁₆COOH |
| 20:0 | Arachidic acid | eicosanoic acid | CH₃(CH₂)₁₈COOH |
| 22:0 | Behenic acid | Docosanoic acid | CH₃(CH₂)₂₀COOH |

TABLE 1B

| Symbol | Common Name | Systematic name | Structure |
|---|---|---|---|
| 18:1 | Oleic acid | 9-Octadecenoic acid | CH₃(CH₂)₇CH═CH(CH₂)₇COOH |
| 16:1 | Palmitoleic acid | 9-Hexadecenoic acid | CH₃(CH₂)₅CH═CH(CH₂)₇COOH |
| 18:2 | Linoleic acid | 9,12-Octadecadienoic acid | CH₃(CH₂)₄(CH═CHCH₂)₂(CH₂)₆COOH |
| 20:4 | Arachidonic acid | 5,8,11,14-Eicosatetraenoic acid | CH₃(CH₂)₄(CH═CHCH₂)₄(CH₂)₂COOH |

The cationic lipids and the PC can be used in a ration of 30-70 to 70-30. In one embodiment, the ratio is 40-60 to 60-40. In another embodiment, the ratio is about 50:50. In a further embodiment, the cationic lipid is DOTAP and the PC is DMPC.

The composition of the present invention may optionally comprise CpG sequence: DCs express Toll like receptors (TLRs) that play a fundamental role in the recognition of immune response. CpG, unmethylated cytosine-phosphorothioate-guanine has been shown to promote interaction with toll like receptors and promote Th1 type immune response (23, 31). In particular CpG interacts with TLR-9, an intracellular receptor and the internalization of CpG by lipofection has been shown to produce enhanced levels of I1-12 and down stream responses.

In another embodiment, the lipids used in the liposomes can be mannosylated thereby increasing uptake by DCs. The cationic lipid and/or the PC or PC may be mannosylated. The use of this lipid may not be necessary for intra dermal route of administration as the DC's present in skin (Langerhans cells) lack mannose receptor (20)

The composition of the present invention can also optionally include Lipid A or other bacterial lipopolysaccharides to increase the immuno adjuvancy. Once the targeting and intracellular delivery of antigenic peptides is achieved, T-cell activation is required for immune response. However, this requires a co-stimulatory signal to activate DCs and to migrate into lymphoid organs. In the lymphoid organ DCs present antigens to naive T cells. The bacterial lipo polysaccharides such as Lipid A have been shown to induce DCs to mature into stimulatory APC's. Therefore, Lipid A can be included in the formulation as immuno adjuvant to stimulate DCs for a potent immune response. An example of a suitable range of concentration of Lipid A is 1 to 100 μg/ml. An example of a suitable concentration is about 50 μg/ml.

The size of the liposomal particles is such that most of them will not be taken up by macrophages and thereby eliminated from the circulation. Thus, in one embodiment, at least 50% of the liposomes are less than 120 nm. In other embodiments, at least 60, 70, 80 or 90% of the liposomes are less than 120 nm. In a further embodiment, greater than 91, 92, 93, 94, 95, 96 97, 98, 99% of the particles are less than 120 nm. In a further embodiment, the particles are greater than 30 nm. Thus, the particles are typically between 30 and 120 nm. In one embodiment, the average diameter of the particles is between 60 and 70 nm. In another embodiment, the average diameter is about 65 nm.

The antigen for loading DCs are typically anchored in the bilayer of the liposomes as described herein such that it is suitable for presentation. In one embodiment, the trigger loading procedure is used. Briefly, the protein is unfolded under controlled conditions to expose hydrophobic domains. This reduces solubility in aqueous compartment and promotes the hydrophobic interaction between unfolded protein and lipid bilayer. In another embodiment, the antigen to be presented to the DCs can be covalently linked to the molecules of the liposomes. Thus, antigens can be linked to liposomes by conjugation reaction between the antigen and lipid, or preformed liposomes containing modified and reactive phosphatidylethanolamine (PE) can be used. The approach of covalently linking the antigen onto preformed liposomes often leads to homogeneous size distributions. The antigen linked PE can then be used to form liposomes. In this approach, the antigen linked to inner bilayer will also be achieved. Irrespective of the procedure used, a cross linking agent between PE and antibody will be used. Heteroftinctional cross linking agents (such as N-succininidyl 4-(p-maleiomidophenyl) butyrate (SMBP)) can be used to modify amino groups on the PE and this maleimide-modified lipid or on preformed liposome can be used to link 1H6Ig. The amine group on the protein can be used to introduce sulfhydryl group or alternatively endogenous sulfhydryl can also be used. For example, the reactive amines on the Lysine can be used to introduce a sulfhydryl group using N-succinyimidyl 3-(2-pyridylthio) propionic acid (SPDP) to antibody linked to PDP and can be treated with dithioreitol (DTT) to link a sulfhydryl group on amines. The maleimide modified lipid or liposomes can be treated with reduced PDP-antigen to obtain antigen conjugated lipid or liposomes. Thus, in this embodiment, the liposomes will complise a cationic lipid, PC and PE. The concentration of PE is in the range of 0.5 mol % to 10 mol %.

This invention is useful for facilitating the presentation of any antigen by DCs. For example, this invention can be useful for presentation of tumor antigens, and in particular, B cell tumor antigens. In general tumor antigens are known to have low immunogenicity and this invention will aid in increasing the immunone response by increasing the uptake by DCs. In one embodiment, tumor antigen is a B cell tumor antigen. B cell tumors typically secrete immunoglobulins and therefore, the secreted immunoglobulin or peptides (such as Vh peptides) produced from the immunoglobulins can be used for the liposomal preparations. Such peptides are known in the art (see Lou et al., 2004).

Administration of the composition to an individual can be done by routine methods. In one embodiment, the composition can be administered by a standard route, such as, but not limited to, subcutaneous, intramuscular or intravenous injection. An ex vivo administration can also be carried out. For example, DCs isolated from a patient can be incubated with the lipid nano particle composition and the DCs then administered back to the individual.

The feasibility of the present method was demonstrated in an animal model. A B cell (1H6) tumor and its tumor-associated immunoglobulin (1H6Ig) or 1H6Ig V_{H} peptides were used as the tumor antigen. Both the 1H6Ig protein, and the 1H6Ig V_{H} peptides, are known to be only weakly immunogenic. By using the method of the present invention, we were able to demonstrate that a significant T-cell response as measured by interferon-gamma (IFN-γ), is observed with the composition of the present invention.

### EXAMPLE 1

Preparation, characterization and evaluation of LINAP: A composition comprising lipid nano particles containing 1H6Ig was prepared. Required amount of DOTAP and DMPC was dissolved in chloroform and the solvent was evaporated to form thin film around a round bottomed flask. The film was dispersed in aqueous solution and vortexed at 25° C. for 15 min. The lipidic solution was extruded through series of polycarbonate membranes (0.4, 0.2, 0.08 and 0.05 um) to form LINAP. The size of the LINAP was measured using quasi elastic light scattering and the results indicated that the particle size was around 65 nm (FIG. 2). The intensity of the scattered light was fitted to Gaussain distribution (□² of 0.29). The physico chemical properties of the LINAP were investigated following the encapsulation of other components such as 1H6Ig. The protein was encapsulated into LINAP using conventional procedure. The lipid film was rehydrated using phosphate buffered saline and was vortexed above the phase transition temperature. In addition, the samples were subjected to repeated temperature cycles of 4 and 40° C. The MLVs thus formed was filtered through series of polycarbonate filter to obtain particle size in the range of 65 nm (FIG. 2). The free protein was separated from LINAP associated 1H6Ig by dextran centrifugation gradient. The concentration of 1H6Ig in each band was determined using either by spectral or by routine protein quantitation assays. The encapsulation efficiency was around 40±4%. The morphology of the LINAP containing 1H6Ig was investigated using negative stain transmission electron micrograph (FIG. 2A). The location and topology of the 1H6Ig in the LINAP bilayer was determined using fluorescence studies (FIG. 2B). The 1H6Ig encapsulated in lumen and hydrophobic region of the bilayer will be shielded from acrylamide, a collisional quencher of Trp fluorescence. As is clear from FIG. 2B, the fluorescence emission of 1H6Ig is quenched in the presence of 0.5 M acrylamide whereas 1H6Ig loaded in LINAP is shielded. This is further confirmed from the emission maxima of LINAP loaded 1H6Ig. The LINAP loaded 1H6Ig showed a blue shifted emission maxima compared to free 1H6Ig (subjected similar processing stress) that is accompanied by enhancement in fluorescence intensity. Such a shift is generally obtained for a hydrophobic location of the protein indicating that the protein is located in the hydrophobic bilayer compartment.

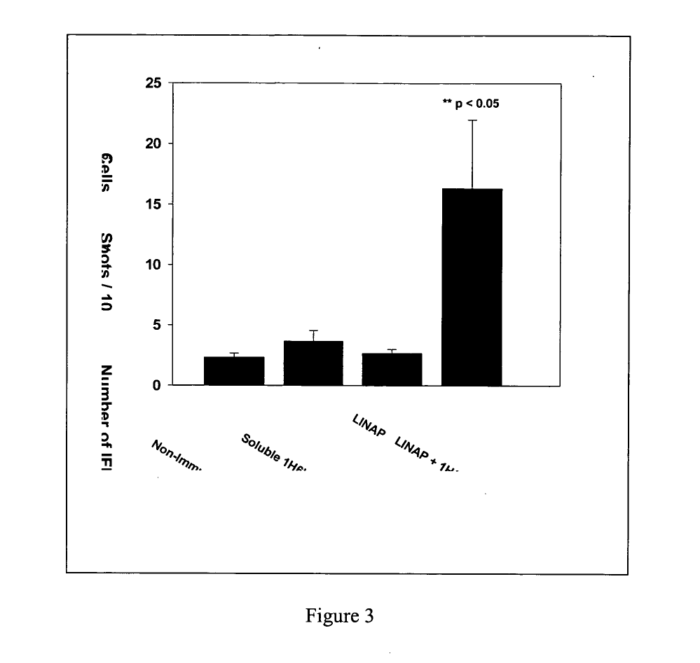

The antigen loaded LINAP induces T-cell responses: In order to develop LINAP as DC based vaccine, T-cell based immune response is very critical. Experimental procedure: The LINAP containing 1H6Ig was prepared using conventional thin film method as described in the previous section. The antigen association/encapsulation procedure and characterization of these particles are described in FIG. 2. The association/encapsulation efficiency was found to be 40±4% and the physical characterization data indicated that the antigen is intercalated within the bilayer and suggests the possibility of luminal location of fraction of the antigen. BALB/c mice were vaccinated intraperitoneally as on days 1 and 7 with 1H6Ig antigen and LINAP associated 1H6Ig. As control, LINAP with no 1H6Ig was also administered. The splenocytes were prepared from the immunized mice and stimulated for 5 days in vitro (IVS) with irradiated 1H6 tumor cells (grown in serum-free medium). The splenocytes were harvested and added to IFN-γ ELISPOT wells. Data Analysis: Each bar represents the mean spot number of triplicates±SEM with 10⁵ splenocytes initially seeded per well and the data was analyzed using one-way ANOVA followed by Dunnet's post hoc analysis. Results and Interpretation: As clear from the FIG. 3, the T-cell response measured as Interferon gamma response is much higher for LINAP loaded 1H6Ig compared to the administration of soluble antigen or unimmunized or LINAP alone. The data clearly indicates that the antigen loaded in LINAP induces higher T-cell responses

References:

1. T. F. Gretenand E. M. Jaffee. Cancer vaccines. J Clin Oncol 17: 1047-60 (1999).
2. D. C. Linehan, P. S. Goedegebuure, and T. J. Eberlein. Vaccine therapy for cancer. Ann Surg Oncol 3: 219-28 (1996).
3. D. R. Madden. The three-dimensional structure of peptide-MHC complexes. Annu Rev Immunol 13: 587-622 (1995).
4. M. Gotoh, H. Takasu, K. Harada, and T. Yamaoka. Development of HLA-A2402/K(b) transgenic mice. Int J Cancer 100: 565-70 (2002).
5. B. R. Minev, F. L. Chavez, and M. S. Mitchell. Cancer vaccines: novel approaches and new promise. Pharmacol Ther 81: 121-39 (1999).
6. B. W. Anderson, G. E. Peoples, J. L. Murray, M. A. Gillogly, D. M. Gershenson, and C. G. Ioannides. Peptide priming of cytolytic activity to HER-2 epitope 369-377 in healthy individuals. Clin Cancer Res 6: 4192-200 (2000).
7. K. L. Knutson, K. Schiffman, M. A. Cheever, and M. L. Disis. Immunization of cancer patients with a HER-2/neu, HLA-A2 peptide, p369-377, results in short-lived peptide-specific immunity. Clin Cancer Res 8: 1014-8 (2002).
8. O. J. Finn. Cancer vaccines: between the idea and the reality. Nat Rev Immunol 3: 630-41 (2003).
9. L. H. Brinckerhoff, V. V. Kalashnikov, L. W. Thompson, G. V. Yamshchikov, R. A. Pierce, H. S. Galavotti, V. H. Engelhard, and C. L. Slingluff, Jr. Terminal modifications inhibit proteolytic degradation of an immunogenic MART-1(27-35) peptide: implications for peptide vaccines. Int J Cancer 83: 326-34 (1999).
10. C. D. Partidos, P. Vohra, D. Jones, G. Farrar, and M. W. Steward. CTL responses induced by a single immunization with peptide encapsulated in biodegradable microparticles. J Immunol Methods 206: 143-51 (1997).
11. C. R. Alving, V. Koulchin, G. M. Glenn, and M. Rao. Liposomes as carriers of peptide antigens: induction of antibodies and cytotoxic T lymphocytes to conjugated and unconjugated peptides. Immunol Rev 145: 5-31 (1995).
12. J. J. Bergers, W. Den Otter, H. F. Dullens, C. T. Kerkvliet, and D. J. Crommelin. Interleukin-2-containing liposomes: interaction of interleukin-2 with liposomal bilayers and preliminary studies on application in cancer vaccines. Pharm Res 10: 1715-21 (1993).
13. S. V. Balasubramanian, J. Bruenn, and R. M. Straubinger. Liposomes as formulation excipients for protein pharmaceuticals: a model protein study. Pharm Res 17: 344-50 (2000).
14. P. R. Dal Monte and F. C. Szoka, Jr. Antigen presentation by B cells and macrophages of cytochrome c and its antigenic fragment when conjugated to the surface of liposomes. Vaccine 7: 401-8 (1989).
15. M. Rao, N. M. Wassef, C. R. Alving, and U. Krzych. Intracellular processing of liposome-encapsulated antigens by macrophages depends upon the antigen. Infect Immun 63: 2396-402 (1995).
16. M. Rao, S. W. Rothwell, N. M. Wassef, R. E. Pagano, and C. R. Alving. Visualization of peptides derived from liposome-encapsulated proteins in the trans-Golgi area of macrophages. Immunol Lett 59: 99-105 (1997).
17. R. Ignatius, K. Mahnke, M. Rivera, K. Hong, F. Isdell, R. M. Steinman, M. Pope, and L. Stamatatos. Presentation of proteins encapsulated in sterically stabilized liposomes by dendritic cells initiates CD8(+) T-cell responses in vivo. Blood 96: 3505-13 (2000).
18. C. Esche, M. R. Shurin, and M. T. Lotze. The use of dendritic cells for cancer vaccination. Curr Opin Mol Ther 1: 72-81 (1999).
19. J. Banchereau and R. M. Steinman. Dendritic cells and the control of immunity. Nature 392: 245-52 (1998).
20. C. Watts and S. Amigorena. Antigen traffic pathways in dendritic cells. Traffic 1: 312-7 (2000).
21. K. Inaba, S. Turley, F. Yamaide, T. lyoda, K. Mahnke, M. Inaba, M. Pack, M. Subklewe, B. Sauter, D. Sheff, M. Albert, N. Bhardwaj, I. Mellman, and R. M. Steinman. Efficient presentation of phagocytosed cellular fragments on the major histocompatibility complex class II products of dendritic cells. J Exp Med 188: 2163-73 (1998).
22. C. Foged, C. Arigita, A. Sundblad, W. Jiskoot, G. Storm, and S. Frokjaer. Interaction of dendritic cells with antigen-containing liposomes: effect of bilayer composition. Vaccine 22: 1903-13 (2004).
23. Y. Suzuki, D. Wakita, K. Chamoto, Y. Narita, T. Tsuji, T. Takeshima, H. Gyobu, Y. Kawarada, S. Kondo, S. Akira, H. Katoh, H. Ikeda, and T. Nishimura. Liposome-encapsulated CpG oligodeoxynucleotides as a potent adjuvant for inducing type 1 innate immunity. Cancer Res 64: 8754-60 (2004).
24. M. Whitmore, S. Li, and L. Huang. LPD lipopolyplex initiates a potent cytokine response and inhibits tumor growth. Gene Ther 6: 1867-75 (1999).
25. M. M. Whitmore, S. Li, L. Falo, Jr., and L. Huang. Systemic administration of LPD prepared with CpG oligonucleotides inhibits the growth of established pulmonary metastases by stimulating innate and acquired antitumor immune responses. Cancer Immunol Immunother 50: 503-14 (2001).
26. Y. E. Rahman, E. A. Cemy, K. R. Patel, E. H. Lau, and B. J. Wright. Differential uptake of liposomes varying in size and lipid composition by parenchymal and kupffer cells of mouse liver. Life Sci 31: 2061-71 (1982).
27. C. Oussoren, J. Zuidema, D. J. Crommelin, and G. Storm. Lymphatic uptake and biodistribution of liposomes after subcutaneous injection. II. Influence of liposomal size, lipid compostion and lipid dose. Biochim Biophys Acta 1328: 261-72 (1997).
28. R. B. Campbell, S. V. Balasubramanian, and R. M. Straubinger. Phospholipid-cationic lipid interactions: influences on membrane and vesicle properties. Biochim Biophys Acta 1512: 27-39 (2001).
29. R. M. Straubinger, N. Duzgunes, and D. Papahadjopoulos. pH-sensitive liposomes mediate cytoplasmic delivery of encapsulated macromolecules. FEBS Lett 179: 148-54 (1985).
30. W. Li, F. Nicol, and F. C. Szoka, Jr. GALA: a designed synthetic pH-responsive amphipathic peptide with applications in drug and gene delivery. Adv Drug Deliv Rev 56: 967-85 (2004).
31. C. J. Melief, S. H. Van Der Burg, R. E. Toes, F. Ossendorp, and R. Offringa. Effective therapeutic anticancer vaccines based on precision guiding of cytolytic T lymphocytes. Immunological Reviews 188: 177-82 (2002).
32. D. Reisser, A. Pance, and J. F. Jeannin. Mechanisms of the antitumoral effect of lipid A. Bioessays 24: 284-9 (2002).
33. S. W. Rothwell, N. M. Wassef, C. R. Alving, and M. Rao. Proteasome inhibitors block the entry of liposome-encapsulated antigens into the classical MHC class I pathway. Immunol Lett 74: 141-52 (2000).
34. M. Rao, S. W. Rothwell, N. M. Wassef, A. B. Koolwal, and C. R. Alving. Trafficking of liposomal antigen to the trans-Golgi of murine macrophages requires both liposomal lipid and liposomal protein. Exp Cell Res 246: 203-11 (1999).
35. L. BenMohamed, A. Thomas, and P. Druilhe. Long-term multiepitopic cytotoxic-T-lymphocyte responses induced in chimpanzees by combinations of Plasmodium falciparum liver-stage peptides and lipopeptides. Infect Immun 72: 4376-84 (2004).
36. S. K. Ghosh and R. B. Bankert. Generation of somatic variants of a B cell hybrid mediated by a non-cytolytic L3T4+ idiotype-specific T cell. J Immunol 142: 409-15 (1989).
37. Q. Lou, R. J. Kelleher, Jr., A. Sette, J. Loyall, S. Southwood, R. B. Bankert, and S. H. Bernstein. Germ line tumor-associated immunoglobulin VH region peptides provoke a tumor-specific immune response without altering the response potential of normal B cells. Blood 104: 752-9 (2004).

1. A composition comprising liposomes, wherein the liposomes comprise:
a) a cationic lipid;
b) phosphatidyl choline (PC); and
c) an antigen;
wherein the size of at least 50% of the liposomes is less than 120 nm.

2. The composition of claim 1, wherein the liposomes are from 30 nm to 120 nm in diameter.

3. The composition of claim 1, wherein the average size of liposomes is between 60-70 nm in diameter.

4. The composition of claim 1, wherein at least 90% of the liposomes are less than 120 nm in diameter.

5. The composition of claim 1, wherein the composition further comprises phosphatidyl ethanolamine (PE) and at least some of the antigen molecules are covalently attached to PE.

6. The composition of claim 5, wherein the PE is present between 0.5 mol % to 10 mol %.

7. The composition of claim 1, wherein the acyl chains on the cationic lipid are between 16 and 22 carbons in length.

8. The composition of claim 7, wherein the acyl chains are saturated.

9. The composition of claim 1, wherein the acyl chains of PC are 12-22 carbons in length.

10. The composition of claim 9, wherein the PC is dimyristoylphosphatidylcholine.

11. The composition of claim 1, wherein the acyl chains in the cationic lipid have from 12-22 carbons.

12. The composition of claim 11, wherein the cationic lipid is 1,2-Diacyl-3-Trimethylammonium-Propane (TAP); 1,2-Diacyl-3-Dimethylammonium-Propane (DAP); and/or 1,2-Diacyl-sn-Glycero-3-Ethylphosphocholine (EPC).

13. The composition of claim 12, wherein the cationic lipid is 1,2-Dioleyl-3-Trimethylammonium-Propane (DOTAP); 1,2-Dioleyl-3-Dimethylammonium-Propane (DODAP). Other examples include 18:1 EPC, 18:0 EPC and/or 14:0-18:1 EPC.

14. The composition of claim 1, wherein the cationic lipid and PC are present in a ratio of from 40:60 to 60:40.

15. The composition of claim 1, wherein the cationic lipid is DOTAP and the PC is DMPC and the DOTAP and PC are present in a ratio of about 50:50.

16. The composition of claim 1, wherein the composition further comprises CpG DNA.

17. The composition of claim 1, wherein the composition further comprises Lipid A or a bacterial lipopolysaccharide.

18. The composition of claim 1, wherein the cationic lipid, and/or PC is mannosylated.

19. A method for increasing immune response to an antigen in an individual, comprising administration of the composition of claim 1 to the individual, wherein the administration results in an increased immune response to the antigen compared to the immune response of the antigen alone.

20. The method of claim 19, wherein the composition of claim 1 is incubated with dendritic cells obtained from the individual prior to being administered to the individual.
